(19) United States
(12) Patent Application Publication
(10) Pub. No.: US 2017/0087193 A1
(43) Pub. Date: Mar. 30, 2017

(54) ANTIBACTERIAL ANALGESIC ANTI-INFLAMMATORY SALVE

(71) Applicants: Yorgos D. Marinakis, Rio Rancho, NM (US); Michael Anthony Martinez, Albuquerque, NM (US)

(72) Inventors: Yorgos D. Marinakis, Rio Rancho, NM (US); Michael Anthony Martinez, Albuquerque, NM (US)

(73) Assignee: Miraculite Minerals LLC, Edgewood, NM (US)

(21) Appl. No.: 14/869,934

(22) Filed: Sep. 29, 2015

(51) Int. Cl. A61K 35/644; A61K 36/889; A61K 9/06; A61K 36/28; A61K 9/00; A61K 33/06; A61K 31/355

(52) U.S. Cl. CPC A61K 35/644; A61K 33/06; A61K 36/889; A61K 31/355; A61K 36/28; A61K 9/0014; A61K 9/06

(57) ABSTRACT

An antibacterial analgesic anti-inflammatory salve comprising 62 percent by volume of beeswax; 10 percent by volume of smectite clay that was pushed through a number 200 mesh sieve having openings of 0.0029 inches; and 28 percent by volume of a plurality of antibacterial, antifungal, analgesic and anti-inflammatory organic oils derived from vegetable matter. A method for making an antibacterial analgesic anti-inflammatory salve.

### CROSS-REFERENCE TO RELATED APPLICATIONS

Not applicable.

### STATEMENTS REGARDING FEDERALLY SPONSORED RESEARCH AND DEVELOPMENT

Not applicable.

### BACKGROUND

The embodiments of the present invention comprise an antibacterial analgesic anti-inflammatory salve and a method for making the same.

Information relevant to attempts to address these problems can be found in U.S. Pat. Nos. 3,955,999; 4,883,664; 5,270,042; 5,328,682; 5,719,197; 5,932,230; 6,136,765; 6,365,139; 6,716,418; 6,749,860; 8,021,700; 8,795,735; 8,901,067; 9,044,417; 9,050,259; which are not admitted to be prior art with respect to the present invention by its mention in this Background Section. However, it is desirable to have a better salve than is disclosed in the references.

Beeswax salves typically comprise larger amounts of oil and/or naphtha than beeswax. Ratios of oil and/or naphtha to beeswax typically range from 1:1 to 10:1. In the embodiments of the present invention, that ratio is reversed, to 1:2.2 oils to beeswax. In addition, we add a substantial amount of smectite clay, ground to a specific consistency and in a proportion that facilitate homogeneous distribution of the clay throughout the salve.

### SUMMARY

The embodiments of the present invention are directed to a salve and a method that satisfy the needs set out in the Background section.

The embodiments of the present invention comprise an antibacterial analgesic anti-inflammatory salve comprising:

62 percent by volume of antibacterial beeswax;
10 percent by volume of antibacterial smectite clay that was pushed through a number 200 mesh sieve having openings of 0.0029 inches; and
28 percent by volume of a plurality of antibacterial, antifungal, analgesic and anti-inflammatory organic oils derived from vegetable matter.
A sample formulation is 6 ounces beeswax, 1 ounce smectite clay, and the remaining approximately 2.67 ounces of organic oil.

Optionally, the 28 percent by volume of a plurality of antibacterial, antifungal, analgesic and anti-inflammatory organic oils derived from vegetable matter comprises:

21 percent by volume of antibacterial and antifungal coconut oil;
5 percent by volume of anti-inflammatory Vitamin E oil; and
2 percent by volume of analgesic Arnica oil.
A sample formulation is 2 ounces coconut oil, 0.5 ounce (1 Tablespoon) Vitamin E oil and 0.167 ounce (1 teaspoon) Arnica oil.

The embodiments of the invention further comprise a method for making an antibacterial analgesic anti-inflammatory salve, comprising melting 62 percent by volume of antibacterial beeswax, to form a mixture thereof, slightly cooling said mixture; adding 10 percent by volume of antibacterial smectite clay that was pushed through a number 200 mesh sieve having openings of 0.0029 inches, and 28 percent by volume of a plurality of antibacterial, antifungal, analgesic and anti-inflammatory organic oils derived from vegetable matter, to said slightly cooled mixture and further cooling said mixture to room temperature to form said salve. A sample formulation is 6 ounces beeswax, 1 ounce smectite clay, and the remaining approximately 2.67 ounces of organic oil.

Optionally, the 28 percent by volume of a plurality of antibacterial, antifungal, analgesic and anti-inflammatory organic oils derived from vegetable matter comprises:

21 percent by volume of antibacterial and antifungal coconut oil;
5 percent by volume of anti-inflammatory Vitamin E oil; and
2 percent by volume of analgesic Arnica oil.
A sample formulation is 2 ounces coconut oil, 0.5 ounce (1 Tablespoon) Vitamin E oil and 0.167 ounce (1 teaspoon) Arnica oil.

### Some Advantages of the Embodiments of the Invention

Beeswax and smectite clay naturally have antibacterial properties. Coconut oil naturally has both antibacterial and antifungal properties. Arnica is a natural analgesic. Vitamin E oil natural has anti-inflammatory properties. Beeswax also inhibits salve loss due to perspiration.

These and other advantages will become apparent as the embodiments of the invention are used.

### DESCRIPTION

### Definitions

According to Wikipedia, “Organic oils are produced in remarkable diversity by plants, animals, and other organisms through natural metabolic processes. Lipid is the scientific term for the fatty acids, steroids and similar chemicals often found in the oils produced by living things, while oil refers to an overall mixture of chemicals. Organic oils may also contain chemicals other than lipids, including proteins, waxes (class of compounds with oil-like properties that are solid at common temperatures) and alkaloids. Lipids can be classified by the way that they are made by an organism, their chemical structure and their limited solubility in water compared to oils. They have a high carbon and hydrogen content and are considerably lacking in oxygen compared to other organic compounds and minerals; they tend to be relatively nonpolar molecules, but may include both polar and nonpolar regions as in the case of phospholipids and steroids.” Organic Vitamin E oil can be obtained by vacuum distillation of vegetable fats derived from soy sources. Arnica oil is made from the Arnica montana plant. Coconut oil, or copra oil, is extracted from the kernel or meat of mature coconuts harvested from the coconut palm Cocos nucifera.

Beeswax (Cera alba) is a natural wax produced by honey bees of the genus Apis.

### Description of the Preferred Embodiment and Best Mode

The preferred embodiment of the present invention comprises an antibacterial analgesic anti-inflammatory salve comprising:

62 percent by volume of antibacterial beeswax;
10 percent by volume of antibacterial smectite clay that was pushed through a number 200 mesh sieve having openings of 0.0029 inches; and
28 percent by volume of a plurality of antibacterial, antifungal, analgesic and anti-inflammatory organic oils derived from vegetable matter.

Preferably, the 28 percent by volume of a plurality of antibacterial, antifungal, analgesic and anti-inflammatory organic oils derived from vegetable matter comprises:

21 percent by volume of antibacterial and antifungal coconut oil;
5 percent by volume of anti-inflammatory Vitamin E oil; and
2 percent by volume of analgesic Arnica oil.

The salve can be applied directly to the skin. It can also be poured, when in liquid form, onto the gauze side of a rubber- or plastic-backed gauze pad, which pad with wet or dry salve can then be applied to the skin.

Some advantages of the embodiments of the apparatus were previously enumerated in the Summary section. Every advantageous feature does not need to be incorporated into every embodiment of the apparatus and/or methods.

Although these versions of the invention have been described in considerable detail, other versions are possible. For example, embodiments can comprise combinations of the features described herein, such as combinations of the dependent claims. Therefore, the spirit and scope of the appended claims should not be limited to the description of the versions contained therein.

We claim:

1. An antibacterial analgesic anti-inflammatory salve comprising:
62 percent by volume of antibacterial beeswax;
10 percent by volume of antibacterial smectite clay that was pushed through a number 200 mesh sieve having openings of 0.0029 inches; and
28 percent by volume of a plurality of antibacterial, antifungal, analgesic and anti-inflammatory organic oils derived from vegetable matter.

2. The salve of claim 1, wherein the 28 percent by volume of a plurality of antibacterial, antifungal, analgesic and anti-inflammatory organic oils derived from vegetable matter comprises:
21 percent by volume of antibacterial and antifungal coconut oil;
5 percent by volume of anti-inflammatory Vitamin E oil; and
2 percent by volume of analgesic Arnica oil.

3. A method for making an antibacterial analgesic anti-inflammatory salve, comprising melting 62 percent by volume of antibacterial beeswax, to form a mixture thereof, slightly cooling said mixture; adding 10 percent by volume of antibacterial smectite clay that was pushed through a number 200 mesh sieve having openings of 0.0029 inches, and 28 percent by volume of a plurality of antibacterial, antifungal, analgesic and anti-inflammatory organic oils derived from vegetable matter, to said slightly cooled mixture and further cooling said mixture to room temperature to form said salve.

4. The method of claim 3, wherein the 28 percent by volume of a plurality of antibacterial, antifungal, analgesic and anti-inflammatory organic oils derived from vegetable matter comprises:
21 percent by volume of antibacterial and antifungal coconut oil;
5 percent by volume of anti-inflammatory Vitamin E oil; and
2 percent by volume of analgesic Arnica oil.
